(19) United States
(12) Patent Application Publication
(10) Pub. No.: US 2020/0309727 A1
(43) Pub. Date: Oct. 1, 2020

(54) GAS SENSOR AND SENSOR ELEMENT

(71) Applicant: NGK INSULATORS, LTD., Nagoya (JP)

(72) Inventors: Taku OKAMOTO, Nagoya (JP); Osamu NAKASONE, Inabe (JP); Nobukazu IKOMA, Nagoya (JP); Noriko HIRATA, Nagoya (JP)

(21) Appl. No.: 16/819,813

(22) Filed: Mar. 16, 2020

(30) Foreign Application Priority Data
Mar. 27, 2019 (JP) 2019-059956

(51) Int. Cl. G01N 27/409; G01N 27/407; G01N 27/41; G01N 33/00

(52) U.S. Cl. CPC G01N 27/409; G01N 27/4074; G01N 33/0037; G01N 27/41; G01N 33/0054; G01N 27/4077

(57) ABSTRACT

A gas sensor includes an element body including oxygen-ion-conductive solid electrolyte layers and having a measurement-object gas flow section inside the element body; a main pump cell configured to adjust the oxygen concentration in a first internal cavity; an auxiliary pump cell configured to adjust the oxygen concentration in a second internal cavity; a measurement electrode disposed on an inner peripheral surface of a third internal cavity; and a reference electrode. An inner pump electrode of the main pump cell does not contain a noble metal having a catalytic activity inhibition ability. An auxiliary pump electrode of the auxiliary pump cell contains the noble metal having the catalytic activity inhibition ability.

The present application claims priority to Japanese Patent Application No. 2019-059956, filed on Mar. 27, 2019, the entire contents of which are incorporated herein by reference.

### BACKGROUND OF THE INVENTION

### 1. Field of the Invention

The present invention relates to gas sensors and sensor elements.

### 2. Description of the Related Art

Gas sensors for detecting the concentration of a specific gas such as NOx in a measurement-object gas such as automotive exhaust gas are known in the related art. For example, PTL 1 discloses a gas sensor including a stack of oxygen-ion-conductive solid electrolyte layers and electrodes disposed on the solid electrolyte layers. This gas sensor detects NOx concentration as follows. First, oxygen is pumped from a measurement-object gas flow section inside a sensor element to the outside of the sensor element or from the outside of the sensor element to the measurement-object gas flow section to adjust the oxygen concentration in the measurement-object gas flow section. After the oxygen concentration is adjusted, NOx in the measurement-object gas is reduced. The NOx concentration in the measurement-object gas is detected based on the current that flows through an electrode (measurement electrode) inside the sensor element depending on the oxygen concentration after reduction. PTL 2 discloses a gas sensor for detecting the ammonia concentration in a measurement-object gas. This gas sensor detects the ammonia concentration by oxidizing ammonia with oxygen in the measurement-object gas for conversion into NOx and detecting the concentration of the ammonia-derived NOx in the same manner as in PTL 1.

PTL 1 also discloses that an inner pump electrode, disposed in the measurement-object gas flow section, of a pump cell for adjusting the oxygen concentration is a cermet electrode composed of Pt and ZrO₂ and containing 1% Au. If the inner pump electrode contains Au, it can prevent the inner pump electrode from reducing NOx. On the other hand, PTL 3 discloses that, during the use of such a gas sensor, Au evaporates from the electrode of the pump cell and is deposited on an electrode of a sensor cell for detecting the NOx concentration in the measurement-object gas and that, as a result, the NOx concentration detection accuracy decreases.

### CITATION LIST

### Patent Literature

PTL 1: JP 2014-190940 A

PTL 2: JP 2011-039041 A

PTL 3: JP 6447568 B

### SUMMARY OF THE INVENTION

If the inner pump electrode reduces NOx, the NOx concentration detection accuracy decreases, which is why the inner pump electrode needs to contain Au. On the other hand, as described above, if the inner pump electrode contains Au, a problem arises in that the NOx concentration detection accuracy decreases during the use of the gas sensor.

The present invention has been made in view of the foregoing problem. A primary object of the invention is to maintain the specific gas concentration detection accuracy for a long period of time.

After conducting intensive research in order to solve the foregoing problem, the inventors have found that, if the atmosphere around the inner main pump electrode is not a low-oxygen-concentration atmosphere, little NOx is reduced by the inner main pump electrode even if the inner main pump electrode does not contain Au. Accordingly, the inventors have found that the inner main pump electrode need not contain Au, which has been believed to be essential in the related art, if the concentration of a specific gas in a measurement-object gas that is not a low-oxygen-concentration atmosphere is measured, thus completing the present invention.

A gas sensor according to the present invention includes:

an element body including an oxygen-ion-conductive solid electrolyte layer and having a measurement-object gas flow section inside the element body for introduction and flow of a measurement-object gas;

a main pump cell configured to pump oxygen out of a first internal cavity of the measurement-object gas flow section to adjust an oxygen concentration in the first internal cavity;

an auxiliary pump cell configured to pump oxygen out of a second internal cavity downstream of the first internal cavity of the measurement-object gas flow section to adjust an oxygen concentration in the second internal cavity;

a measurement electrode disposed on an inner peripheral surface of a measurement chamber downstream of the second internal cavity of the measurement-object gas flow section;

a reference electrode disposed inside the element body for introduction of a reference gas serving as a reference for detection of a concentration of a specific gas in the measurement-object gas;

a measurement-voltage detection unit configured to detect a measurement voltage between the reference electrode and the measurement electrode; and

a specific gas concentration detection unit configured to acquire, based on the measurement voltage, a detection value depending on oxygen derived from the specific gas in the measurement chamber and to detect, based on the detection value, the concentration of the specific gas in the measurement-object gas,

wherein the main pump cell includes an inner main pump electrode disposed in the first internal cavity,

the auxiliary pump cell includes an inner auxiliary pump electrode disposed in the second internal cavity,

the inner main pump electrode, the inner auxiliary pump electrode, and the measurement electrode each contain a catalytically active noble metal,

the inner main pump electrode does not contain a noble metal having a catalytic activity inhibition ability to inhibit catalytic activity of the catalytically active noble metal on the specific gas, and

the inner auxiliary pump electrode contains the noble metal having the catalytic activity inhibition ability.

In this gas sensor, the main pump cell and the auxiliary pump cell each pump out oxygen to adjust the oxygen concentration in the measurement-object gas introduced into the measurement-object gas flow section. Thus, the measurement-object gas whose oxygen concentration has been adjusted reaches the measurement chamber. This gas sensor acquires, based on the measurement voltage, the detection value depending on oxygen derived from the specific gas in the measurement chamber and detects, based on the acquired detection value, the concentration of the specific gas in the measurement-object gas. Here, even if the inner main pump electrode does not contain the noble metal having the catalytic activity inhibition ability (e.g., Au), little of the specific gas or the oxide derived from the specific gas is reduced by the inner main pump electrode if the measurement-object gas introduced into the measurement-object gas flow section is not a low-oxygen-concentration atmosphere. Thus, the gas sensor according to the present invention has sufficient specific gas concentration detection accuracy. In addition, because the inner main pump electrode does not contain the noble metal having the catalytic activity inhibition ability, the evaporation and deposition of the noble metal on the measurement electrode can be inhibited during the use of the gas sensor. Thus, the gas sensor according to the present invention can maintain its specific gas concentration detection accuracy for a long period of time when used as a gas sensor for measuring the concentration of a specific gas in a measurement-object gas that is not a low-oxygen-concentration atmosphere. That is, the gas sensor according to the present invention is particularly suitable for the measurement of the concentration of a specific gas in a measurement-object gas that is not a low-oxygen-concentration atmosphere.

Here, “not contain the noble metal having the catalytic activity inhibition ability” refers to being substantially free of the noble metal having the catalytic activity inhibition ability; that is, the noble metal having the catalytic activity inhibition ability may be present as an incidental impurity.

Here, if the specific gas is an oxide, “oxygen derived from the specific gas in the measurement chamber” may be oxygen produced when the specific gas itself is reduced in the measurement chamber. If the specific gas is a non-oxide, “oxygen derived from the specific gas in the measurement chamber” may be oxygen produced when a gas obtained by converting the specific gas into an oxide is reduced in the measurement chamber. In addition, the specific gas concentration detection unit may acquire, as the detection value, a measurement pump current that flows when oxygen derived from the specific gas in the measurement chamber is pumped out of the measurement chamber based on the measurement voltage so that the oxygen concentration in the measurement chamber is at a predetermined low concentration. The element body may be a stack including a plurality of oxygen-ion-conductive solid electrolyte layers stacked on top of each other.

The inner auxiliary pump electrode of the gas sensor according to the present invention may contain Au as the noble metal having the catalytic activity inhibition ability.

A sensor element according to the present invention includes:

an element body including an oxygen-ion-conductive solid electrolyte layer and having a measurement-object gas flow section inside the element body for introduction and flow of a measurement-object gas;

a main pump cell configured to pump oxygen out of a first internal cavity of the measurement-object gas flow section to adjust an oxygen concentration in the first internal cavity;

an auxiliary pump cell configured to pump oxygen out of a second internal cavity downstream of the first internal cavity of the measurement-object gas flow section to adjust an oxygen concentration in the second internal cavity;

a measurement electrode disposed on an inner peripheral surface of a measurement chamber downstream of the second internal cavity of the measurement-object gas flow section; and

a reference electrode disposed inside the element body for introduction of a reference gas serving as a reference for detection of a concentration of a specific gas in the measurement-object gas,

wherein the main pump cell includes an inner main pump electrode disposed in the first internal cavity,

the auxiliary pump cell includes an inner auxiliary pump electrode disposed in the second internal cavity,

the inner main pump electrode, the inner auxiliary pump electrode, and the measurement electrode each contain a catalytically active noble metal,

the inner main pump electrode does not contain a noble metal having a catalytic activity inhibition ability to inhibit catalytic activity of the catalytically active noble metal on the specific gas, and

the inner auxiliary pump electrode contains the noble metal having the catalytic activity inhibition ability.

As with the above-described gas sensor according to the present invention, this sensor element can be used to detect the concentration of the specific gas in the measurement-object gas. In addition, as with the above-described gas sensor according to the present invention, the inner main pump electrode of this sensor element does not contain the noble metal having the catalytic activity inhibition ability, whereas the inner auxiliary pump electrode contains the noble metal having the catalytic activity inhibition ability. Thus, the sensor element according to the present invention can maintain its specific gas concentration detection accuracy for a long period of time when used to detect the concentration of a specific gas in a measurement-object gas that is not a low-oxygen-concentration atmosphere. That is, the sensor element according to the present invention is particularly suitable for the measurement of the concentration of a specific gas in a measurement-object gas that is not a low-oxygen-concentration atmosphere.

### BRIEF DESCRIPTION OF THE DRAWINGS

FIG. 1 is a schematic sectional view of a gas sensor 100.

FIG. 2 is a block diagram showing electrical connections between a controller 90 and individual cells.

FIG. 3 is a graph showing the relationship between NO concentration and pump current Ip2 for gas sensors of Experimental Examples 1 to 4.

FIG. 4 is a schematic sectional view of a sensor element 201.

### DETAILED DESCRIPTION OF THE INVENTION

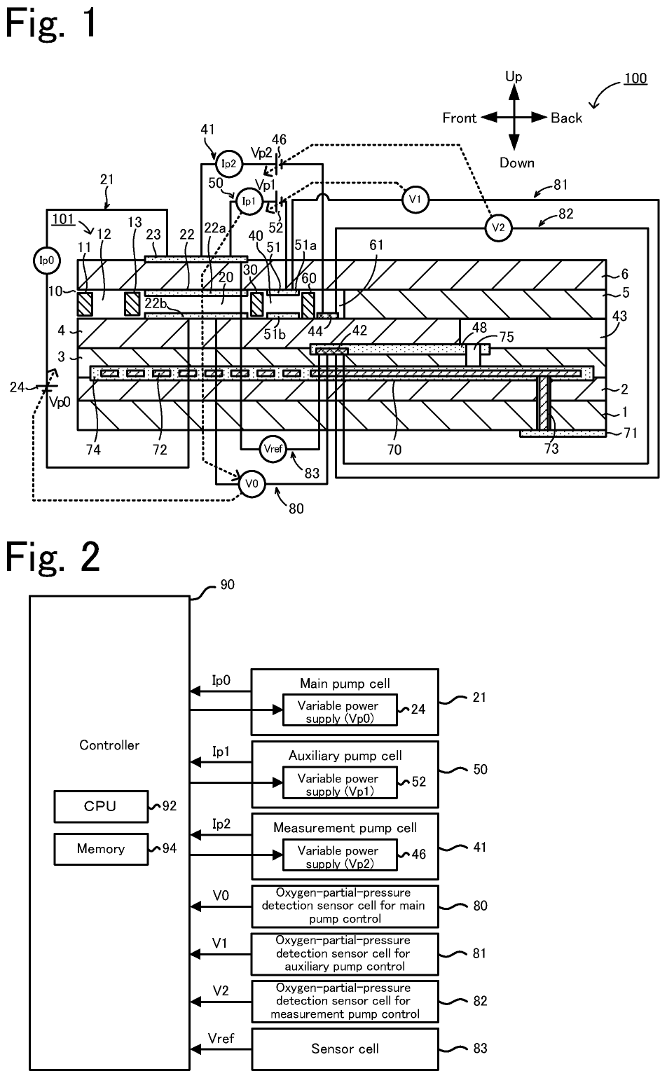

Embodiments of the present invention will now be described with reference to the drawings. FIG. 1 is a schematic sectional view showing, in outline, an example configuration of a gas sensor 100 according to one embodiment of the present invention. FIG. 2 is a block diagram showing electrical connections between a controller 90 and individual cells. This gas sensor 100 is attached to, for example, a pipe such as an exhaust gas pipe of an internal combustion engine such as a gasoline engine or a diesel engine. The gas sensor 100 detects the concentration of a specific gas, such as NOx or ammonia, in an exhaust gas from an internal combustion engine, which serves as a measurement-object gas. In this embodiment, the gas sensor 100 is configured to measure NOx concentration as the concentration of the specific gas. The gas sensor 100 includes a sensor element 101 having an elongated rectangular parallelepiped shape, individual cells 21, 41, 50, and 80 to 83, each including part of the sensor element 101, and a controller 90 configured to control the overall gas sensor 100.

The sensor element 101 is an element including a layered body in which six layers, namely a first substrate layer 1, a second substrate layer 2, a third substrate layer 3, a first solid electrolyte layer 4, a spacer layer 5, and a second solid electrolyte layer 6, are layered in this order from the bottom side, as viewed in the drawing. Each of the six layers is formed of an oxygen-ion-conductive solid electrolyte layer containing, for example, zirconia (ZrO₂). The solid electrolyte forming these six layers is dense and gastight. This sensor element 101 is manufactured, for example, by stacking ceramic green sheets corresponding to the individual layers on top of each other, for example, after predetermined processing and circuit pattern printing, and then firing the stacked ceramic green sheets so that they are combined together.

A gas inlet 10, a first diffusion-rate limiting section 11, a buffer space 12, a second diffusion-rate limiting section 13, a first internal cavity 20, a third diffusion-rate limiting section 30, a second internal cavity 40, a fourth diffusion-rate limiting section 60, and a third internal cavity 61 are formed adjacent to each other so as to communicate in the above order between the lower surface of the second solid electrolyte layer 6 and the upper surface of the first solid electrolyte layer 4 on the front end side (on the left end side in FIG. 1) of the sensor element 101.

The gas inlet port 10, the buffer space 12, the first internal space 20, the second internal space 40, and the third internal space 61 constitute a space within the sensor element 101. The space is provided in such a manner that a portion of the spacer layer 5 is hollowed out. The top of the space is defined by the lower surface of the second solid electrolyte layer 6, the bottom of the space is defined by the upper surface of the first solid electrolyte layer 4, and sides of the space are defined by side surfaces of the spacer layer 5.

The first diffusion-rate limiting section 11, the second diffusion-rate limiting section 13, and the third diffusion-rate limiting section 30 are each provided as two laterally elongated slits (i.e., the longitudinal direction of the openings is perpendicular to the figure). The fourth diffusion-rate limiting section 60 is provided as a single laterally elongated slit (i.e., the longitudinal direction of the opening is perpendicular to the figure) formed as a clearance under the lower surface of the second solid electrolyte layer 6. The section extending from the gas inlet 10 to the third internal cavity 61 is also referred to as “measurement-object gas flow section”.

A reference gas introduction space 43 is disposed between the upper surface of the third substrate layer 3 and the lower surface of the spacer layer 5 at a position farther away from the front end side than the measurement-object gas flow section. The reference gas introduction space 43 is defined at both sides by the side surfaces of the first solid electrolyte layer 4. As an example of a reference gas for NOx concentration measurement, air is introduced into the reference gas introduction space 43.

An air introduction layer 48 is a porous ceramic layer. The reference gas is introduced into the air introduction layer 48 through the reference gas introduction space 43. The air introduction layer 48 is formed so as to cover a reference electrode 42.

The reference electrode 42 is formed between the upper surface of the third substrate layer 3 and the first solid electrolyte layer 4. As described above, the air introduction layer 48 leading to the reference gas introduction space 43 is disposed around the reference electrode 42. As described later, the reference electrode 42 can be used to measure the oxygen concentrations (oxygen partial pressures) in the first internal cavity 20, the second internal cavity 40, and the third internal cavity 61. The reference electrode 42 is formed as a porous cermet electrode (e.g., a cermet electrode composed of Pt and ZrO₂).

The gas inlet 10 of the measurement-object gas flow section is open to the external space. The measurement-object gas is taken from the external space through the gas inlet 10 into the sensor element 101. The first diffusion-rate limiting section 11 creates a predetermined diffusion resistance to the measurement-object gas taken through the gas inlet 10. The buffer space 12 is provided to guide the measurement-object gas introduced from the first diffusion-rate limiting section 11 into the second diffusion-rate limiting section 13. The second diffusion-rate limiting section 13 creates predetermined diffusion resistance to the measurement-object gas introduced from the buffer space 12 into the first internal cavity 20. When the measurement-object gas is introduced from outside the sensor element 101 into the first internal cavity 20, the measurement-object gas, which is rapidly taken through the gas inlet 10 into the sensor element 101 due to pressure variations in the measurement-object gas in the external space (pulsations in exhaust pressure if the measurement-object gas is automotive exhaust gas), is not directly introduced into the first internal cavity 20. Rather, the measurement-object gas is introduced into the first internal cavity 20 after concentration variations in the measurement-object gas are eliminated through the first diffusion-rate limiting section 11, the buffer space 12, and the second diffusion-rate limiting section 13. Thus, there are almost negligible concentration variations in the measurement-object gas introduced into the first internal cavity 20. The first internal cavity 20 is provided as a space for adjusting the oxygen partial pressure in the measurement-object gas introduced through the second diffusion-rate limiting section 13. This oxygen partial pressure is adjusted by the operation of a main pump cell 21.

The main pump cell 21 is an electrochemical pump cell composed of an inner pump electrode 22 having a ceiling electrode portion 22a disposed over substantially an entire portion of the lower surface of the second solid electrolyte layer 6 that faces the first internal cavity 20, an outer pump electrode 23 disposed on a region of the upper surface of the second solid electrolyte layer 6 that corresponds to the ceiling electrode portion 22a so as to be exposed to the external space, and a portion of the second solid electrolyte layer 6 that is located between the inner pump electrode 22 and the outer pump electrode 23.

The inner pump electrode 22 is formed on portions of the upper and lower solid electrolyte layers (the second solid electrolyte layer 6 and the first solid electrolyte layer 4) that define the first internal cavity 20 and portions of the spacer layer 5 that give the sidewalls of the first internal cavity 20. Specifically, the ceiling electrode portion 22a is formed on a portion of the lower surface of the second solid electrolyte layer 6 that gives the ceiling surface of the first internal cavity 20. A bottom electrode portion 22b is formed on a portion of the upper surface of the first solid electrolyte layer 4 that gives the bottom surface of the first internal cavity 20. Side electrode portions (not shown) are formed on portions of the sidewall surfaces (inner surfaces) of the spacer layer 5 that form both sidewalls of the first internal cavity 20 so as to join together the ceiling electrode portion 22a and the bottom electrode portion 22b. Thus, the inner pump electrode 22 is provided as a tunnel-like structure in the area where the side electrode portions are disposed.

The inner pump electrode 22 and the outer pump electrode 23 are formed as porous cermet electrodes (e.g., cermet electrodes composed of Pt and ZrO₂).

In the main pump cell 21, the desired pump voltage Vp0 is applied between the inner pump electrode 22 and the outer pump electrode 23 so that a pump current Ip0 flows between the inner pump electrode 22 and the outer pump electrode 23 in either a positive or negative direction. Thus, oxygen can be pumped from the first internal cavity 20 to the external space or from the external space to the first internal cavity 20.

To detect the oxygen concentration (oxygen partial pressure) in the atmosphere in the first internal cavity 20, the inner pump electrode 22, the second solid electrolyte layer 6, the spacer layer 5, the first solid electrolyte layer 4, the third substrate layer 3, and the reference electrode 42 form an electrochemical sensor cell, namely, an oxygen-partial-pressure detection sensor cell 80 for main pump control.

The oxygen concentration (oxygen partial pressure) in the first internal cavity 20 can be detected from the electromotive force V0 measured in the oxygen-partial-pressure detection sensor cell 80 for main pump control. In addition, the pump current Ip0 is controlled by performing feedback control of the pump voltage Vp0 of a variable power supply 24 so that the electromotive force V0 is constant. Thus, the oxygen concentration in the first internal cavity 20 can be maintained at a predetermined constant value.

The third diffusion-rate limiting section 30 creates a predetermined diffusion resistance to the measurement-object gas whose oxygen concentration (oxygen partial pressure) has been controlled in the first internal cavity 20 by the operation of the main pump cell 21 and guides the measurement-object gas into the second internal cavity 40.

The second internal cavity 40 is provided as a space for further adjusting, using an auxiliary pump cell 50, the oxygen concentration (oxygen partial pressure) of the measurement-object gas introduced through the third diffusion-rate limiting section 30 after the oxygen partial pressure is adjusted in advance in the first internal cavity 20. Thus, the oxygen concentration in the second internal cavity 40 can be maintained at a constant value with high accuracy so that the gas sensor 100 can measure the NOx concentration with high accuracy.

The auxiliary pump cell 50 is an auxiliary electrochemical pump cell composed of an auxiliary pump electrode 51 having a ceiling electrode portion 51a disposed over substantially an entire portion of the lower surface of the second solid electrolyte layer 6 that faces the second internal cavity 40, the outer pump electrode 23 (the outer electrode is not limited to the outer pump electrode 23, but may be any suitable electrode outside the sensor element 101), and the second solid electrolyte layer 6.

This auxiliary pump electrode 51 is provided in the second internal cavity 40 as a tunnel-like structure similar to the inner pump electrode 22 disposed in the first internal cavity 20. Specifically, the ceiling electrode portion 51a is formed on a portion of the second solid electrolyte layer 6 that gives the ceiling surface of the second internal cavity 40. A bottom electrode portion 51b is formed on a portion of the first solid electrolyte layer 4 that gives the bottom surface of the second internal cavity 40. Side electrode portions (not shown) are formed on portions of both sidewall surfaces of the spacer layer 5 that give the sidewalls of the second internal cavity 40 so as to join together the ceiling electrode portion 51a and the bottom electrode portion 51b. Thus, the auxiliary pump electrode 51 is provided as a tunnel-like structure.

In the auxiliary pump cell 50, the desired voltage Vp1 is applied between the auxiliary pump electrode 51 and the outer pump electrode 23. Thus, oxygen can be pumped from the atmosphere in the second internal cavity 40 to the external space or from the external space to the second internal cavity 40.

To control the oxygen partial pressure in the atmosphere in the second internal cavity 40, the auxiliary pump electrode 51, the reference electrode 42, the second solid electrolyte layer 6, the spacer layer 5, the first solid electrolyte layer 4, and the third substrate layer 3 form an electrochemical sensor cell, namely, an oxygen-partial-pressure detection sensor cell 81 for auxiliary pump control.

The auxiliary pump cell 50 performs pumping using a variable power supply 52 whose voltage is controlled based on the electromotive force V1 detected in the oxygen-partial-pressure detection sensor cell 81 for auxiliary pump control. Thus, the oxygen partial pressure in the atmosphere in the second internal cavity 40 can be controlled to a partial pressure that has substantially no effect on NOx measurement.

In addition to this, a pump current Ip1 is used for electromotive force control of the oxygen-partial-pressure detection sensor cell 80 for main pump control. Specifically, the pump current Ip1 is input as a control signal to the oxygen-partial-pressure detection sensor cell 80 for main pump control to control the electromotive force V0 so that the gradient of the oxygen partial pressure in the measurement-object gas introduced from the third diffusion-rate limiting section 30 into the second internal cavity 40 remains constant. When the gas sensor 100 is used as a NOx sensor, the oxygen concentration in the second internal cavity 40 is maintained at a constant value of about 0.001 ppm by the operation of the main pump cell 21 and the auxiliary pump cell 50.

The fourth diffusion-rate limiting section 60 creates predetermined diffusion resistance to the measurement-object gas whose oxygen concentration (oxygen partial pressure) has been controlled in the second internal cavity 40 by the operation of the auxiliary pump cell 50 and guides the measurement-object gas into the third internal cavity 61. The fourth diffusion-rate limiting section 60 functions to limit the amount of NOx flowing into the third internal cavity 61.

The third internal cavity 61 is provided as a space for processing associated with the measurement of the nitrogen oxide (NOx) concentration in the measurement-object gas introduced through the fourth diffusion-rate limiting section 60 after the oxygen concentration (oxygen partial pressure) is adjusted in advance in the second internal cavity 40. NOx concentration measurement is mainly performed in the third internal cavity 61 by the operation of a measurement pump cell 41.

The measurement pump cell 41 measures the NOx concentration in the measurement-object gas in the third internal cavity 61. The measurement pump cell 41 is an electrochemical pump cell composed of a measurement electrode 44 disposed on a portion of the upper surface of the first solid electrolyte layer 4 that faces the third internal cavity 61, the outer pump electrode 23, the second solid electrolyte layer 6, the spacer layer 5, and the first solid electrolyte layer 4. The measurement electrode 44 also functions as a NOx reduction catalyst to reduce NOx present in the atmosphere in the third internal cavity 61.

The measurement pump cell 41 pumps out oxygen produced by the decomposition of nitrogen oxide in the atmosphere around the measurement electrode 44. The amount of oxygen produced can be detected as a pump current Ip2.

To detect the oxygen partial pressure around the measurement electrode 44, the first solid electrolyte layer 4, the third substrate layer 3, the measurement electrode 44, and the reference electrode 42 form an electrochemical sensor cell, namely, an oxygen-partial-pressure detection sensor cell 82 for measurement pump control. A variable power supply 46 is controlled based on the electromotive force V2 detected in the oxygen-partial-pressure detection sensor cell 82 for measurement pump control.

The measurement-object gas guided into the second internal cavity 40 flows through the fourth diffusion-rate limiting section 60 at controlled oxygen partial pressure to reach the measurement electrode 44 in the third internal cavity 61. The nitrogen oxide in the measurement-object gas around the measurement electrode 44 is reduced to produce oxygen (2NO→N₂+O₂). The resulting oxygen is pumped by the measurement pump cell 41. During this process, the voltage Vp2 of the variable power supply 46 is controlled so that the electromotive force V2 detected in the oxygen-partial-pressure detection sensor cell 82 for measurement pump control is constant. Because the amount of oxygen produced around the measurement electrode 44 is proportional to the nitrogen oxide concentration in the measurement-object gas, the nitrogen oxide concentration in the measurement-object gas is calculated from the pump current Ip2 through the measurement pump cell 41.

In addition, the second solid electrolyte layer 6, the spacer layer 5, the first solid electrolyte layer 4, the third substrate layer 3, the outer pump electrode 23, and the reference electrode 42 form an electrochemical sensor cell 83. The oxygen partial pressure in the measurement-object gas outside the gas sensor 100 can be detected from the electromotive force Vref generated by the sensor cell 83.

In the gas sensor 100 having the foregoing configuration, the main pump cell 21 and the auxiliary pump cell 50 are operated to supply a measurement-object gas whose oxygen partial pressure has been maintained at a constant low value (a value that has substantially no effect on NOx measurement) to the measurement pump cell 41. Thus, the NOx concentration in the measurement-object gas can be detected based on the pump current Ip2 that flows as oxygen produced by the reduction of NOx is pumped out by the measurement pump cell 41 substantially in proportion to the NOx concentration in the measurement-object gas.

To increase the oxygen ion conductivity of the solid electrolyte, the sensor element 101 further includes a heater section 70 that functions as a temperature regulator to heat and maintain the temperature of the sensor element 101. The heater section 70 includes a heater connector electrode 71, a heater 72, a through-hole 73, a heater insulating layer 74, and a pressure relief vent 75.

The heater connector electrode 71 is formed in contact with the lower surface of the first substrate layer 1. The heater connector electrode 71 is connected to an external power supply so that the heater section 70 can be externally powered.

The heater 72 is an electrical resistor formed between the second substrate layer 2 and the third substrate layer 3. The heater 72 is connected to the heater connector electrode 71 through the through-hole 73. The heater 72 is externally powered through the heater connector electrode 71 to generate heat, thereby heating and maintaining the temperature of the solid electrolyte forming the sensor element 101.

The heater 72 is embedded over the entire region from the first internal cavity 20 to the third internal cavity 61 so that the temperature of the entire sensor element 101 can be adjusted to a temperature that activates the solid electrolyte.

The heater insulating layer 74 is an insulating layer covering the upper and lower surfaces of the heater 72 and formed of an insulator such as alumina. The heater insulating layer 74 is formed in order to ensure electrical insulation between the second substrate layer 2 and the heater 72 and electrical insulation between the third substrate layer 3 and the heater 72.

The pressure relief vent 75 extends through the third substrate layer 3 and the air introduction layer 48 so as to communicate with the reference gas introduction space 43. The pressure relief vent 75 is formed in order to mitigate an increase in internal pressure due to a temperature increase in the heater insulating layer 74.

The inner pump electrode 22, the auxiliary pump electrode 51, and the measurement electrode 44 each contain a catalytically active noble metal. The catalytically active noble metal may be, for example, at least one of Pt, Rh, Ir, Ru, and Pd. The outer pump electrode 23 and the reference electrode 42 also contain the catalytically active noble metal. The auxiliary pump electrode 51 further contains a noble metal having a catalytic activity inhibition ability to inhibit the catalytic activity of the catalytically active noble metal on the specific gas. Thus, the auxiliary pump electrode 51 exhibits a weakened ability to reduce the NOx component in the measurement-object gas. The noble metal having the catalytic activity inhibition ability may be, for example, Au. In contrast, the inner pump electrode 22 does not contain the noble metal having the catalytic activity inhibition ability. The measurement electrode 44 preferably does not contain the noble metal having the catalytic activity inhibition ability. The outer pump electrode 23 and the reference electrode 42 also preferably do not contain the noble metal having the catalytic activity inhibition ability. The electrodes 22, 23, 42, 44, and 51 are each preferably formed of a cermet containing a noble metal and an oxygen-ion-conductive oxide (e.g., ZrO₂). The electrodes 22, 23, 42, 44, and 51 are each preferably porous. In this embodiment, the electrodes 22, 23, 42, and 44 are porous cermet electrodes composed of Pt and ZrO₂, and the auxiliary pump electrode 51 is a porous cermet electrode composed of Pt and ZrO₂ and containing 1% Au.

The controller 90 is a microprocessor including, for example, a CPU 92 and a memory 94. The controller 90 receives the electromotive force V0 detected in the oxygen-partial-pressure detection sensor cell 80 for main pump control, the electromotive force V1 detected in the oxygen-partial-pressure detection sensor cell 81 for auxiliary pump control, the electromotive force V2 detected in the oxygen-partial-pressure detection sensor cell 82 for measurement pump control, the electromotive force Vref detected in the sensor cell 83, the pump current Ip0 detected in the main pump cell 21, the pump current Ip1 detected in the auxiliary pump cell 50, and the pump current Ip2 detected in the measurement pump cell 41. The controller 90 transmits control signals to the variable power supply 24 of the main pump cell 21, the variable power supply 52 of the auxiliary pump cell 50, and the variable power supply 46 of the measurement pump cell 41.

The controller 90 performs feedback control of the pump voltage Vp0 of the variable power supply 24 so that the electromotive force V0 is at the target value (referred to as the target value V0*) (i.e., so that the oxygen concentration in the first internal cavity 20 is at a constant target concentration). Thus, the pump current Ip0 varies depending on the oxygen concentration in the measurement-object gas.

The controller 90 also performs feedback control of the voltage Vp1 of the variable power supply 52 so that the electromotive force V1 is at a constant value (referred to as the target value V1*) (i.e., so that the oxygen concentration in the second internal cavity 40 is at a predetermined low oxygen concentration that has substantially no effect on NOx measurement). In addition to this, the controller 90 sets the target value V0* of the electromotive force V0 based on the pump current Ip1 that flows with the voltage Vp1 so that the pump current Ip1 is at a constant value (referred to as the target value Ip1*) (feedback control). Thus, the gradient of the oxygen partial pressure in the measurement-object gas introduced from the third diffusion-rate limiting section 30 into the second internal cavity 40 remains constant. In addition, the oxygen partial pressure in the atmosphere in the second internal cavity 40 is controlled to a low partial pressure that has substantially no effect on NOx measurement.

The controller 90 also performs feedback control of the voltage Vp2 of the variable power supply 46 so that the electromotive force V2 is at a constant value (referred to as the target value V2*) (i.e., so that the oxygen concentration in the third internal cavity 61 is at a predetermined low concentration). Thus, oxygen is pumped out of the third internal cavity 61 so that the concentration of oxygen produced by the reduction of NOx in the measurement-object gas is substantially zero in the third internal cavity 61. The controller 90 acquires the pump current Ip2 as the detection value depending on oxygen derived from the specific gas (here, NOx) in the third internal cavity 61 and calculates the NOx concentration in the measurement-object gas based on the pump current Ip2.

The memory 94 stores a relational formula between pump current Ip2 and NOx concentration, for example, in the form of a linear function. This relational formula can be experimentally determined in advance.

An example of the use of the thus-configured gas sensor 100 will be described below. It is assumed that the CPU 92 of the controller 90 is operating to control the pump cells 21, 41, and 50 described above and to acquire the voltages V0, V1, V2, and Vref from the sensor cells 80 to 83 described above. In this state, when the measurement-object gas is introduced from the gas inlet 10, the measurement-object gas first passes through the first diffusion-rate limiting section 11, the buffer space 12, and the second diffusion-rate limiting section 13 in the above order to reach the first internal cavity 20. The oxygen concentration in the measurement-object gas is then adjusted in the first internal cavity 20 by the main pump cell 21 and in the second internal cavity 40 by the auxiliary pump cell 50. After adjustment, the measurement-object gas reaches the third internal cavity 61. The CPU 92 detects the NOx concentration in the measurement-object gas based on the acquired pump current Ip2 and the relational formula stored in the memory 94.

Here, as described above, the inner pump electrode 22 does not contain the noble metal having the catalytic activity inhibition ability, whereas the auxiliary pump electrode 51 contains the noble metal having the catalytic activity inhibition ability. The reason for this will be explained. The inventors provided gas sensors of Experimental Examples 1 to 4 that had the same configuration as the gas sensor 100 but differed in the presence or absence of Au in the inner pump electrode 22 and the auxiliary pump electrode 51, as shown in Table 1. For all of Experimental Examples 1 to 4, the inner pump electrode 22 and the auxiliary pump electrode 51 were porous cermet electrodes composed of a noble metal and ZrO₂. In Table 1, “0.8” means that the electrodes contained Pt and Au as noble metals and that the mass percentage of Au relative to Pt in the electrodes is 0.8 wt %. In Table 1, “-” means that the electrodes contained only Pt as a noble metal and did not contain Au.

TABLE 1

Mass percentage of Au relative to Pt
in the electrodes[wt %]

| | inner pump | auxiliary pump |
|---|---|---|
| | electrode | electrode |
| Experimental Example 1 | 0.8 | 0.8 |
| Experimental Example 2 | — | — |
| Experimental Example 3 | — | 0.8 |
| Experimental Example 4 | 0.8 | — |

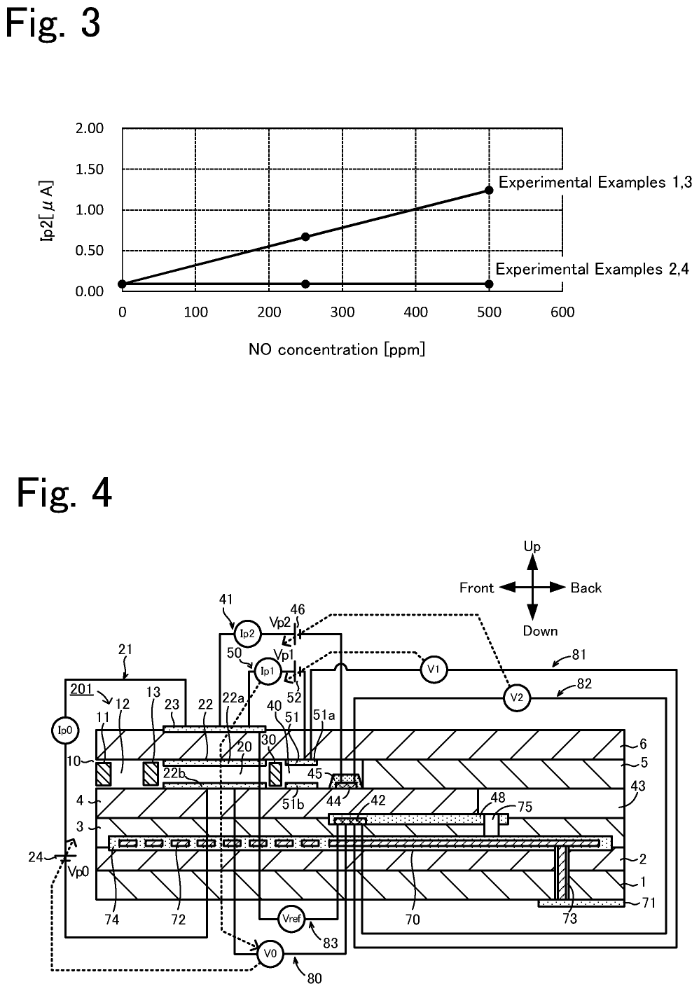

The gas sensors of Experimental Examples 1 to 4 were each investigated for the relationship between the concentration of a specific gas in a measurement-object gas and the pump current Ip2 in the case that the measurement-object gas was not a low-oxygen-concentration atmosphere. As the measurement-object gas, three model gases containing 0 ppm, 250 ppm, and 500 ppm NO as the specific gas component were prepared and used. For all three model gases, nitrogen was used as a base gas, the moisture concentration was adjusted to 3% by volume, and the oxygen concentration was adjusted to 10% by volume. The temperature of the model gases was 250° C. The model gases were allowed to flow through a pipe with a diameter of 20 mm at a flow rate of 50 L/min. The relationship between NO concentration and pump current Ip2 for the gas sensors of Experimental Examples 1 to 4 is shown in Table 2 and FIG. 3.

TABLE 2

| NO | Pump current Ip2[μA] | | | |
|---|---|---|---|---|
| concentration | Experimental | Experimental | Experimental | Experimental |
| [ppm] | Example 1 | Example 2 | Example 3 | Example 4 |
| 0 | 0.09 | 0.09 | 0.09 | 0.09 |
| 250 | 0.67 | 0.09 | 0.67 | 0.09 |
| 500 | 1.24 | 0.09 | 1.24 | 0.09 |

As can be seen from the results shown in Table 2 and FIG. 3, Experimental Examples 2 and 4, in which the auxiliary pump electrode 51 did not contain Au, exhibited little change in Ip2 as the NO concentration was changed, and the pump current Ip2 was almost 0 μA. This is probably because NO was reduced by the catalytic activity of the auxiliary pump electrode 51 before reaching the measurement electrode 44. In contrast, Experimental Examples 1 and 3, in which the auxiliary pump electrode 51 contained Au, exhibited a proportional relationship between NO concentration and Ip2. In addition, the values of Ip2 corresponding to those of NO concentration for Experimental Examples 1 and 3 were almost equal to each other. That is, whether the inner pump electrode 22 contained Au or not did not affect the pump current Ip2. These results indicate that NO is not reduced by the inner pump electrode 22 even if, whereas the auxiliary pump electrode 51 contains Au, the inner pump electrode 22 does not contain Au. From these results, the inventors found that the inner pump electrode 22 need not contain Au if the measurement-object gas is not a low-oxygen-concentration atmosphere. Based on these results, the inner pump electrode 22 of the gas sensor 100 according to this embodiment does not contain Au, whereas the auxiliary pump electrode 51 contains Au. That is, Experimental Example 3 corresponds to the gas sensor 100 according to this embodiment, and therefore, to an example of the gas sensor according to the present invention. Experimental Examples 1, 2, and 4 correspond to comparative examples.

The reason for the results discussed above is believed to be as follows. During the use of the gas sensor 100, the main pump cell 21 and the auxiliary pump cell 50 are controlled as described above by the CPU 92 to pump out oxygen if the measurement-object gas is not a low-oxygen-concentration atmosphere. Thus, the relationship between the oxygen concentrations around the gas inlet 10 and the electrodes in the measurement-object gas flow section is believed to be as follows: (around gas inlet 10)>(around inner pump electrode 22)>(around auxiliary pump electrode 51)>(around measurement electrode 44). That is, the oxygen concentration is higher around the inner pump electrode 22 than around the auxiliary pump electrode 51. NOx is less likely to be reduced at a higher oxygen concentration; therefore, NOx is less likely to be reduced by the inner pump electrode 22 even if the inner pump electrode 22 does not contain the noble metal having the catalytic activity inhibition ability (here, Au). On the other hand, NOx is more likely to be reduced by the auxiliary pump electrode 51 because the measurement-object gas reaches the area around the auxiliary pump electrode 51 after oxygen is pumped out by the main pump cell 21. However, the reduction of NOx can be inhibited because the auxiliary pump electrode 51 contains Au. Thus, the gas sensor 100 according to this embodiment has a sufficiently low tendency to reduce NOx before the measurement-object gas reaches the measurement electrode 44 and thus has a sufficient specific gas concentration detection accuracy.

If the inner pump electrode 22 contains Au, Au may evaporate from the inner pump electrode 22 and may be deposited on the measurement electrode 44 during the use of the gas sensor 100. The deposition of Au on the measurement electrode 44 inhibits the catalytic activity of the measurement electrode 44 and thus leads to insufficient reduction of NOx around the measurement electrode 44. As a result, the actual pump current Ip2 decreases compared to the correct pump current Ip2 corresponding to the NOx concentration, thus decreasing the specific gas concentration detection accuracy. In contrast, because the inner pump electrode 22 of the gas sensor 100 according to this embodiment does not contain the noble metal having the catalytic activity inhibition ability, the evaporation of the noble metal can be inhibited during the use of the gas sensor 100, and therefore, the decrease in detection accuracy during use can be reduced.

Thus, the gas sensor 100 according to this embodiment can maintain its specific gas concentration detection accuracy for a long period of time. In contrast, for example, as in Experimental Examples 2 and 4, if the auxiliary pump electrode 51 does not contain Au, the specific gas concentration detection accuracy is already decreased at the point of time when the use of the gas sensor is started. For example, as in Experimental Example 1, if the inner pump electrode 22 contains Au, the specific gas concentration detection accuracy tends to decrease during the use of the gas sensor. That is, the durability of the gas sensor decreases.

Although the auxiliary pump electrode 51 contains Au, Au in the auxiliary pump electrode 51 has a relatively low tendency to evaporate. This will be explained. The evaporation of Au from an electrode as described above is more likely to occur at a higher oxygen concentration. For example, in the case of an electrode containing Pt and Au, Pt is more likely to be oxidized to form PtO₂ at a higher oxygen concentration. PtO₂ has a higher tendency to evaporate than Pt because PtO₂ has a higher saturated vapor pressure than Pt. As Pt evaporates in the form of PtO₂, the remaining Au also tends to evaporate. This is because Au alone has a higher saturated vapor pressure than a Pt—Au alloy. In contrast, Au in the auxiliary pump electrode 51 has a relatively low tendency to evaporate because, as described above, the oxygen concentration is lower around the auxiliary pump electrode 51. Thus, the decrease in detection accuracy during the use of the gas sensor 100 as described above is less likely to occur even if the auxiliary pump electrode 51 contains Au.

Here, the correspondences between the elements of this embodiment and the elements of the present invention are shown below. The stack of six layers of this embodiment, namely, the first substrate layer 1, the second substrate layer 2, the third substrate layer 3, the first solid electrolyte layer 4, the spacer layer 5, and the second solid electrolyte layer 6 that are stacked in the above order, corresponds to an element body of the present invention. The first internal cavity 20 corresponds to a first internal cavity. The main pump cell 21 corresponds to a main pump cell. The second internal cavity 40 corresponds to a second internal cavity. The auxiliary pump cell 50 corresponds to an auxiliary pump cell. The third internal cavity 61 corresponds to a measurement chamber. The measurement electrode 44 corresponds to a measurement electrode. The reference electrode 42 corresponds to a reference electrode. The oxygen-partial-pressure detection sensor cell 82 for measurement pump control corresponds to a measurement-voltage detection unit. The pump current Ip2 corresponds to a detection value. The CPU 92 of the controller 90 corresponds to a specific gas concentration detection unit. The inner pump electrode 22 corresponds to an inner main pump electrode. The auxiliary pump electrode 51 corresponds to an inner auxiliary pump electrode.

The inner pump electrode 22 of the above-described gas sensor 100 according to this embodiment does not contain the noble metal having the catalytic activity inhibition ability (e.g., Au); however, little specific gas is reduced by the inner pump electrode 22 if the measurement-object gas introduced into the measurement-object gas flow section is not a low-oxygen-concentration atmosphere. In addition, because the inner pump electrode 22 does not contain the noble metal having the catalytic activity inhibition ability, the evaporation and deposition of the noble metal on the measurement electrode 44 can be inhibited during the use of the gas sensor 100. Thus, the gas sensor 100 can maintain its specific gas concentration detection accuracy for a long period of time when used in applications in which the concentration of a specific gas in a measurement-object gas that is not a low-oxygen-concentration atmosphere is measured. That is, the gas sensor 100 is particularly suitable for the measurement of the concentration of a specific gas in a measurement-object gas that is not a low-oxygen-concentration atmosphere.

It should be understood that the present invention is not limited to the embodiment described above in any way, but can be practiced in various embodiments that fall within the technical scope of the invention.

Although the second diffusion-rate limiting section 13 is present between the buffer space 12 and the first internal cavity 20 in the embodiment described above, the present invention is not limited thereto. For example, the second diffusion-rate limiting section 13 may be omitted, and the buffer space 12 and the first internal cavity 20 may form a single space.

Although the gas sensor 100 according to the embodiment described above is configured to detect NOx concentration as the concentration of the specific gas, the present invention is not limited thereto. Rather, the gas sensor 100 may be configured to detect the concentration of another oxide as the concentration of the specific gas. If the specific gas is an oxide, oxygen is produced when the specific gas itself is reduced in the third internal cavity 61, as in the embodiment described above; therefore, the CPU 92 can acquire the detection value depending on the oxygen and detect the concentration of the specific gas. Alternatively, the specific gas may be a non-oxide such as ammonia. If the specific gas is a non-oxide, the specific gas is converted into an oxide (e.g., ammonia is converted into NO). Because oxygen is produced when the converted gas is reduced in the third internal cavity 61, the CPU 92 can acquire the detection value depending on the oxygen and detect the concentration of the specific gas. For example, because the inner pump electrode 22 contains the catalytically active noble metal described above, the specific gas can be converted into an oxide in the first internal cavity 20. Because ammonia is converted into NO as an oxide, ammonia concentration measurement is basically performed on the same principle as NOx concentration measurement.

Although the sensor element 101 of the gas sensor 100 according to the embodiment described above includes the first internal cavity 20, the second internal cavity 40, and the third internal cavity 61, the present invention is not limited thereto. For example, as in the case of a sensor element 201 in FIG. 4, the third internal cavity 61 may be omitted. In the sensor element 201 according to the modification shown in FIG. 4, the gas inlet 10, the first diffusion-rate limiting section 11, the buffer space 12, the second diffusion-rate limiting section 13, the first internal cavity 20, the third diffusion-rate limiting section 30, and the second internal cavity 40 are formed adjacent to each other so as to communicate in the above order between the lower surface of the second solid electrolyte layer 6 and the upper surface of the first solid electrolyte layer 4. The measurement electrode 44 is disposed on the upper surface of the first solid electrolyte layer 4 in the second internal cavity 40. The measurement electrode 44 is covered by a fourth diffusion-rate limiting section 45. The fourth diffusion-rate limiting section 45 is a porous film of a ceramic such as alumina (Al₂O₃). As with the fourth diffusion-rate limiting section 60 of the embodiment described above, the fourth diffusion-rate limiting section 45 functions to limit the amount of NOx flowing into the measurement electrode 44. The fourth diffusion-rate limiting section 45 also functions as a protective film for the measurement electrode 44. The ceiling electrode portion 51a of the auxiliary pump electrode 51 is formed so as to extend over the measurement electrode 44. As in the embodiment described above, the thus-configured sensor element 201 can detect NOx concentration, for example, based on the pump current Ip2. In this case, the area around the measurement electrode 44 functions as a measurement chamber.

Although the outer pump electrode 23 functions as an outer main pump electrode of the main pump cell 21, as an outer auxiliary pump electrode of the auxiliary pump cell 50, and as an outer measurement electrode of the measurement pump cell 41 in the embodiment described above, the present invention is not limited thereto. Besides the outer pump electrode 23, one or more of an outer main pump electrode, an outer auxiliary pump electrode, and an outer measurement electrode may be disposed outside the element body so as to contact the measurement-object gas.

Although the element body of the sensor element 101 according to the embodiment described above is a stack including a plurality of solid electrolyte layers (the layers 1 to 6), the present invention is not limited thereto. The element body of the sensor element 101 may include at least one oxygen-ion-conductive solid electrolyte layer and have a measurement-object gas flow section inside the element body. For example, the layers 1 to 5 other than the second solid electrolyte layer 6 in FIG. 1 may be layers formed of materials other than solid electrolytes (e.g., alumina layers). In this case, the electrodes of the sensor element 101 may be disposed on the second solid electrolyte layer 6. For example, the measurement electrode 44 in FIG. 1 may be disposed on the lower surface of the second solid electrolyte layer 6. In addition, the reference gas introduction space 43 may be disposed in the spacer layer 5 rather than in the first solid electrolyte layer 4. The air introduction layer 48 may be disposed between the second solid electrolyte layer 6 and the spacer layer 5 rather than between the first solid electrolyte layer 4 and the third substrate layer 3. The reference electrode 42 may be disposed on the lower surface of the second solid electrolyte layer 6 on the rear side of the third internal cavity 61.

Although the controller 90 in the embodiment described above sets the target value V0* of the electromotive force V0 based on the pump current Ip1 so that the pump current Ip1 is at the target value Ip1* (feedback control) and performs feedback control of the pump voltage Vp0 so that the electromotive force V0 is at the target value V0*, other control may also be performed. For example, the controller 90 may perform feedback control of the pump voltage Vp0 based on the pump current Ip1 so that the pump current Ip1 is at the target value Ip1*. That is, the acquisition of the electromotive force V0 from the oxygen-partial-pressure detection sensor cell 80 for main pump control and the setting of the target value V0* may be omitted, and the controller 90 may control the pump voltage Vp0 (and thereby control the pump current Ip0) directly based on the pump current Ip1.

Although not described in the embodiment described above, the gas sensor 100 is preferably used for the measurement of the concentration of a specific gas in a measurement-object gas having an oxygen concentration of more than 0.1% by volume. That is, “measurement-object gas that is not a low-oxygen-concentration atmosphere” may be a measurement-object gas having an oxygen concentration of more than 0.1% by volume. In Experimental Examples 1 to 4 described above, the oxygen concentration in the measurement-object gas reaching the second internal cavity 40 (=oxygen concentration at exit of third diffusion-rate limiting section 30) was detected to be 0.1% by volume. This suggests that the auxiliary pump electrode 51 needed to contain Au because the oxygen concentration was not more than 0.1% by volume around the auxiliary pump electrode 51. On the other hand, as can be seen from the above-described relationship between the oxygen concentrations around the electrodes in the measurement-object gas flow section, the oxygen concentration was more than 0.1% by volume around the inner pump electrode 22. This explains why NO was not reduced even though the inner pump electrode 22 did not contain Au. Thus, if the measurement-object gas has an oxygen concentration of more than 0.1% by volume, the reduction of the specific gas or the oxide derived from the specific gas by the inner pump electrode 22 can be more reliably inhibited even if the inner pump electrode 22 does not contain the noble metal having the catalytic activity inhibition ability. That is, the need for the inner pump electrode 22 to contain the noble metal having the catalytic activity inhibition ability can be more reliably eliminated. More preferably, the gas sensor 100 is used for the measurement of the concentration of a specific gas in a measurement-object gas having an oxygen concentration of 1% by volume or more. In this case, the need for the inner pump electrode 22 to contain the noble metal having the catalytic activity inhibition ability can be even more reliably eliminated.

In the embodiment described above, the main pump cell 21 may pump oxygen out of the first internal cavity 20 so that the oxygen concentration in the measurement-object gas reaching the second internal cavity 40 is not less than 0.1% by volume. In this case, the likelihood that the oxygen concentration becomes low around the inner pump electrode 22 can be reduced. Thus, the reduction of the specific gas or the oxide derived from the specific gas by the inner pump electrode 22 can be more reliably inhibited if the inner pump electrode 22 does not contain the noble metal having the catalytic activity inhibition ability. The CPU 92 preferably controls the main pump cell 21 to perform such oxygen pumping. For example, the permissible range of the target value V0* described above may be experimentally determined in advance so that the oxygen concentration in the measurement-object gas reaching the second internal cavity 40 is not less than 0.1% by volume. When setting the target value V0* based on the pump current Ip1, the CPU 92 may set the target value V0* within this permissible range.

What is claimed is:

1. A gas sensor comprising:
an element body including an oxygen-ion-conductive solid electrolyte layer and having a measurement-object gas flow section inside the element body for introduction and flow of a measurement-object gas;
a main pump cell configured to pump oxygen out of a first internal cavity of the measurement-object gas flow section to adjust an oxygen concentration in the first internal cavity;
an auxiliary pump cell configured to pump oxygen out of a second internal cavity downstream of the first internal cavity of the measurement-object gas flow section to adjust an oxygen concentration in the second internal cavity;
a measurement electrode disposed on an inner peripheral surface of a measurement chamber downstream of the second internal cavity of the measurement-object gas flow section;
a reference electrode disposed inside the element body and to which a reference gas is to be introduced, the reference gas serving as a reference for detection of a concentration of a specific gas in the measurement-object gas;
a measurement-voltage detection unit configured to detect a measurement voltage between the reference electrode and the measurement electrode; and
a specific gas concentration detection unit configured to acquire, based on the measurement voltage, a detection value depending on oxygen derived from the specific gas in the measurement chamber and to detect, based on the detection value, the concentration of the specific gas in the measurement-object gas,
wherein the main pump cell includes an inner main pump electrode disposed in the first internal cavity,
the auxiliary pump cell includes an inner auxiliary pump electrode disposed in the second internal cavity,
the inner main pump electrode, the inner auxiliary pump electrode, and the measurement electrode each contain a catalytically active noble metal,
the inner main pump electrode does not contain a noble metal having a catalytic activity inhibition ability to inhibit catalytic activity of the catalytically active noble metal on the specific gas, and
the inner auxiliary pump electrode contains the noble metal having the catalytic activity inhibition ability.

2. The gas sensor according to claim 1, wherein
the inner auxiliary pump electrode contains Au as the noble metal having the catalytic activity inhibition ability.

3. A sensor element comprising:
an element body including an oxygen-ion-conductive solid electrolyte layer and having a measurement-object gas flow section inside the element body for introduction and flow of a measurement-object gas;
a main pump cell configured to pump oxygen out of a first internal cavity of the measurement-object gas flow section to adjust an oxygen concentration in the first internal cavity;
an auxiliary pump cell configured to pump oxygen out of a second internal cavity downstream of the first internal cavity of the measurement-object gas flow section to adjust an oxygen concentration in the second internal cavity;
a measurement electrode disposed on an inner peripheral surface of a measurement chamber downstream of the second internal cavity of the measurement-object gas flow section; and
a reference electrode disposed inside the element body and to which a reference gas is to be introduced, the reference gas serving as a reference for detection of a concentration of a specific gas in the measurement-object gas,
wherein the main pump cell includes an inner main pump electrode disposed in the first internal cavity,
the auxiliary pump cell includes an inner auxiliary pump electrode disposed in the second internal cavity,
the inner main pump electrode, the inner auxiliary pump electrode, and the measurement electrode each contain a catalytically active noble metal,
the inner main pump electrode does not contain a noble metal having a catalytic activity inhibition ability to inhibit catalytic activity of the catalytically active noble metal on the specific gas, and
the inner auxiliary pump electrode contains the noble metal having the catalytic activity inhibition ability.
